(19) United States
(12) Patent Application Publication
(10) Pub. No.: US 2024/0316227 A1
(43) Pub. Date: Sep. 26, 2024

(54) Platelet Endothelial Cell Adhesion Molecule-1 (PECAM-1)-Targeted Nanoprobe For Integrated Diagnosis And Treatment, And Preparation Method And Use Thereof

(71) Applicant: UNION HOSPITAL TONGJI MEDICAL COLLEGE HUST, Wuhan (CN)

(72) Inventors: Heng MEI, Wuhan (CN); Yu HU, Wuhan (CN); Zhaozhao CHEN, Wuhan (CN); Lili LUO, Wuhan (CN)

(21) Appl. No.: 18/520,118

(22) Filed: Nov. 27, 2023

(30) Foreign Application Priority Data
Mar. 24, 2023 (CN) 202310295680.3

(51) Int. Cl. A61K 51/10; A61K 51/12

(52) U.S. Cl. CPC A61K 51/1027; A61K 51/1244

(57) ABSTRACT

The present disclosure provides a platelet endothelial cell adhesion molecule-1 (PECAM-1)-targeted nanoprobe for integrated diagnosis and treatment, and a preparation method and use thereof. The preparation method includes the following steps: preparation of radionuclide-labeled PECAM-1_4G6 mAb: subjecting hydrazinonicotinamide (HYNIC) and PECAM-1-4G6 to a reaction in an acidic environment, and filtering a resulting reaction product to allow a reaction with a radionuclide lotion in the presence of a reducing agent to obtain the radionuclide-labeled PECAM-1_4G6 mAb; and preparation of αPECAM-1-AT@HCNPs: adding N,N′-disuccinimidyl carbonate (DSC) into an aqueous solution of HES-CH nanoparticles (HCNPs), mixing evenly, conducting dialysis, and adding the radionuclide-labeled PECAM-1_4G6 mAb to allow a reaction at a room temperature to obtain an aqueous solution of αPECAM-1-HCNPs; and dissolving the αPECAM-1-HCNPs and antithrombin in an equal proportion, such that the antithrombin is adsorbed and loaded onto the @PECAM-1-HCNPs through an electrostatic interaction, and conducting purification to obtain the @PECAM-1-AT@HCNPs.

### TECHNICAL FIELD

The present disclosure belongs to the technical field of nanoprobes, and particularly relates to a platelet endothelial cell adhesion molecule-1 (PECAM-1)-targeted nanoprobe for integrated diagnosis and treatment, and a preparation method and use thereof.

### BACKGROUND

The most serious adverse reaction of a chimeric antigen receptor T-cell immunotherapy (CAR-T) is cytokine release syndrome (CRS), with an incidence rate of approximately 77% to 85%. CRS is caused by the rapid proliferation of CAR-T cells after activation in vivo after reinfusion and excessive cascade release of cytokines (CKs). The CRS may cause patients with high fever, hypotension, dyspnea, coagulation disorders, and end-organ disorders, and even death in severe cases. Currently, CRS grading is mainly determined by clinical manifestations and lacks objective indicators; while commonly used CK monitoring processes have indicator rise that appears later and thresholds that are difficult to quantify. Although existing studies have discovered some of the mechanisms that lead to the occurrence of CRS in CAR-T, no adaptive research has been conducted on same. Whether the monitoring and treatment of CRS can be achieved through corresponding targets has not been studied yet. As a result, it may contribute to the clinical diagnosis and treatment of CRS by exploring early molecular markers and visualization technologies directly related to the CRS and then establishing new means of monitoring and intervention.

### SUMMARY

In view of the above problems, the present disclosure provides a PECAM-1-targeted nanoprobe for integrated diagnosis and treatment, and a preparation method and use thereof. The present disclosure is mainly intended to solve the problems that currently there is no better way to classify CRS, and it is relatively difficult to visually detect early molecular markers directly related to the CRS.

To solve the mentioned technical problems, the present disclosure adopts the following technical solutions:

A first aspect of the present disclosure relates to a preparation method of a PECAM-1-targeted nanoprobe for integrated diagnosis and treatment, including the following steps:
S1, preparation of radionuclide-labeled PECAM-1_4G6 mAb: subjecting hydrazinonicotinamide (HYNIC) and PECAM-1-4G6 to a reaction in an acidic environment, and filtering a resulting reaction product to allow a reaction with a radionuclide lotion in the presence of a reducing agent to obtain the radionuclide-labeled PECAM-1_4G6 mAb; and
S2, preparation of αPECAM-1-AT@HCNPs: adding N,N-disuccinimidyl carbonate (DSC) into an aqueous solution of HES-CH nanoparticles (HCNPs), mixing evenly, conducting dialysis, and adding the radionuclide-labeled PECAM-1_4G6 mAb to allow a reaction at a room temperature to obtain an aqueous solution of αPECAM-1-HCNPs; and dissolving the αPECAM-1-HCNPs and antithrombin in an equal proportion, such that the antithrombin is adsorbed and loaded onto the αPECAM-1-HCNPs through an electrostatic interaction, and conducting purification to obtain the αPECAM-1-AT@HCNPs.

In some cases, the radionuclide is ¹⁷⁷Lu; the reducing agent includes 1-ethyl-3-(3-dimethylaminopropyl)carbodiimide hydrochloride (EDC·HCl) and N-hydroxysuccinimide (NHS); and the reaction is conducted under stirring at a room temperature for 12 h.

In some cases, ¹⁷⁷Lu-labeled PECAM-1_4G6 mAb is added and stirred at a room temperature to allow a reaction to obtain an aqueous solution of ¹⁷⁷Lu-αPECAM-1-HCNPs in step S2. At the room temperature, amidation has a high efficiency while there is a high coupling efficiency.

In some cases, the purification in step S2 specifically includes: after the antithrombin is adsorbed and loaded onto the αPECAM-1-HCNPs through the electrostatic interaction, ultrafiltration is conducted to remove unloaded antithrombin to obtain purified αPECAM-1-AT@HCNPs.

In some cases, the αPECAM-1-HCNPs and the antithrombin are dissolved in ultrapure water in the equal proportion, stirred to allow a reaction to electrostatically load the antithrombin in step S2.

In some cases, a preparation process of the HCNPs includes: dissolving hydroxyethyl starch-cholesterol conjugated amphiphilic polymer (HES-CH) in water and conducting an ultrasonic treatment to obtain a HES-CH aqueous solution; adding chloroform dropwise into the HES-CH aqueous solution, conducting the ultrasonic treatment while adding the chloroform, and conducting rotary evaporation to remove the chloroform to obtain a HCNPs aqueous solution; and subjecting the HCNPs aqueous solution to centrifugation, discarding a resulting precipitate, and fully freeze-drying to obtain the HCNPs.

A second aspect of the present disclosure relates to αPECAM-1-AT@HCNPs prepared by the preparation method. In the present disclosure, although the αPECAM-1-AT@HCNPs can be prepared by the aforementioned method, it is not strictly limited to the aforementioned method, and properties of the αPECAM-1-AT@HCNPs should be considered.

A third aspect of the present disclosure relates to use of the αPECAM-1-AT@HCNPs in preparation of a product for detecting or inhibiting a CRS risk during a CAR-T. The CRS mainly occurs in the CAR-T. The CRS can be detected through the αPECAM-1-AT@HCNPs in the present disclosure, and the CAR-T can be better monitored with risks investigated. The occurrence of CRS in the CAR-T can also be inhibited by the αPECAM-1-AT@HCNPs.

A fourth aspect of the present disclosure relates to use of the αPECAM-1-AT@HCNPs in preparation of a developer for a CRS endothelial injury. The αPECAM-1_AT@HCNPs can also monitor an endothelial injury caused when CRS occurs, and can more intuitively determine a location of the endothelial injury.

A fifth aspect of the present disclosure relates to use of the αPECAM-1-AT@HCNPs in preparation of a drug for inhibiting a CRS systemic inflammatory response disease, where the CRS systemic inflammatory response disease includes a vascular endothelial injury and a vascular barrier dysfunction. Delivering relevant drugs and radionuclide probes by targeting extracellular breakpoints can help promote endothelial reconstruction and CRS alleviation. The present disclosure provides the PECAM-1-targeted nanoprobe for integrated diagnosis and treatment that can promote the endothelial reconstruction and CRS alleviation.

The present disclosure has the following beneficial effects: a radionuclide-labeled single-chain antibody 4G6, which has a half-life (6 d to 8 d) in a time window suitable for a CAR-T, is used to capture microvascular imaging in an early stage of a CRS for a target organ. Meanwhile, nanoparticles with desirable biocompatibility are guided to be loaded with antithrombin (AT) and released accurately in a localized area of the CRS injury, thus inhibiting excessive coagulation activation and reducing inflammations. This strategy can early monitor and intervene in CRS and has a great clinical translation value.

### BRIEF DESCRIPTION OF THE DRAWINGS

FIG. 1 shows an endothelial injury model of CAR-T-related CRS;

FIG. 2 shows a tumor burden of lymphoma-bearing mice detected by small animal in vivo imaging; where (A) ELISA has found that after administration of CAR-T, peripheral blood IL-1β (B) and IL-6 (C) of mice have increased significantly; coagulation index testing has revealed that APTT is prolonged during CRS with statistical differences (D), while there is no significant difference between PT and a control group (E); immunohistochemistry shows that after treatment, a large number of CAR-T home into the tumor and recruit a large number of macrophages (F); and there is H&E staining of various organs in CRS model mice (G);

FIG. 3 shows characterization of a ¹⁷⁷Lu-αPECAM-1-AT@HCNPs nanoprobe; where (A) is an appearance of the nanoprobe observed by TEM; and (B) is particle size distribution of the nanoprobe detected by dynamic light scattering (DLS);

FIG. 4 shows in vivo distribution of the ¹⁷⁷Lu-αPECAM-1-AT@HCNPs nanoprobe in the CRS model mice using DIR alternatively labeled nanoparticles; where (A) is biological distribution of αPECAM-1-DIR@HCNPs in heart, liver, spleen, lung, and kidney of the CRS model mice detected by small animal in vivo imaging; and (B) is quantitative analysis of a fluorescence intensity of αPECAM-1-DIR@HCNPs in various organs;

FIG. 5 shows detection of an inflammatory factor level in the plasma of model mice in each group after intervention with ¹⁷⁷Lu-αPECAM-1-AT@HCNPs;

FIG. 6 shows comparison of leukocyte infiltration in liver, lungs and other organs; evaluation for an impact of intervention measures on an inflammatory response in vivo;

FIG. 7 shows macromolecule leakage assessed using immunofluorescence; and

FIG. 8 shows a model of research ideas involved in the ¹⁷⁷Lu-αPECAM-1-AT@HCNPs nanoprobe.

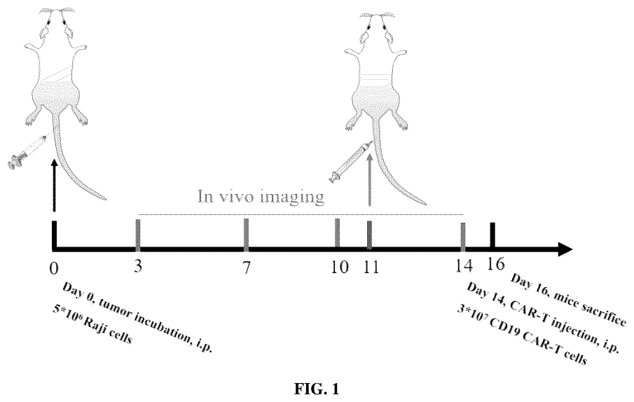

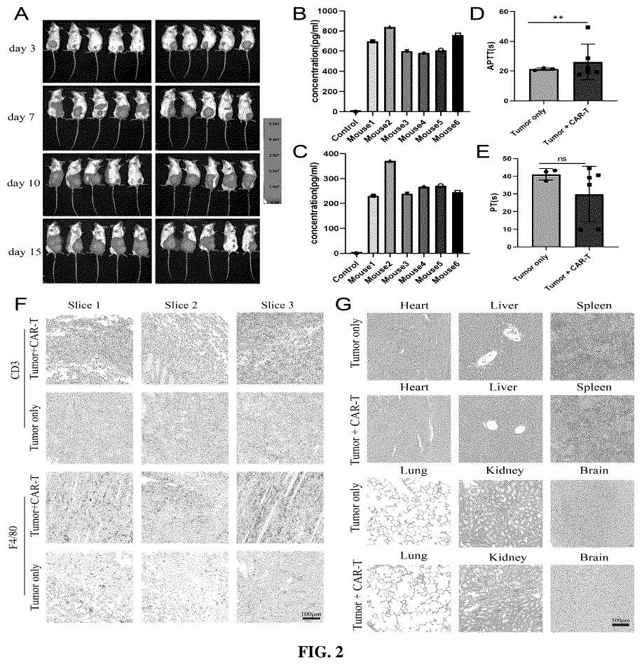

### DETAILED DESCRIPTION OF THE EMBODIMENTS

The present disclosure is further described below:

### (I) Experimental Methods

### 1. Establishment of an Endothelial Injury Model of CAR-T-Related CRS

a. Preparation of CD19 CAR-T cells: peripheral blood was collected from a healthy people, PBMC was separated, CD4⁺ T cells and CD8⁺ T cells were sorted with magnetic beads, and added into cell culture bottles pre-coated with CD3 and CD28 factors, respectively, and the cells were stimulated by IL-2 and IFN-γ to grow and divide faster. The cells were transfected by adding a lentivirus according to a certain MOI, and the lentivirus was washed away after five days. A small number of the cells were subjected to cytokine release detection, killing detection, PCR detection, and flow cytometry to detect a transfection rate, thus determining whether the CAR-T cells had complete functions.

b. Infusion of CAR-T and induction of CRS in mice: several female SCID beige mice aged 6 to 8 weeks were prepared on DO, and 5×10⁶ luciferase-labeled Raji cells were implanted in the abdominal cavity of each mouse; in vivo fluorescence imaging was conducted on D3, D7, D10, and D14 to evaluate tumor burden and exclude mice with extreme burden. Each mouse in the experimental group was intraperitoneally injected with 30×10⁶ CAR-T, and a same amount of normal saline was given in a control group on D14; the CRS could be detected in mice in the experimental group on D16.

2. Preparation and Characterization of PECAM-1-Targeted Nanoprobe for Integrated diagnosis and treatment

a. Preparation of HCNPs by a classic Pickering emulsion solvent evaporation method: 50 mg of HES-CH was dissolved in 50 mL of deionized water, and sonicated with an ultrasonic disruptor for 10 min (at a frequency of 50 Hz, with 2 s of working and 1 s of pausing) to obtain a 1 mg/mL HES-CH aqueous solution; 5 mL of chloroform was slowly added dropwise into the HES-CH aqueous solution, and sonicated with the ultrasonic disruptor for 5 min while adding, to obtain a milky white uniform oil/water mixed solution; the mixed solution was fully subjected to rotary evaporation by a rotary evaporator at 45° C. to remove the chloroform; a resulting HCNPs aqueous solution was centrifuged (5,000 rpm, 10 min), a precipitate was discarded, and freeze-drying was fully conducted to obtain a HCNPs powder. Similarly, DIR@HCNPs were prepared according to the above method: after obtaining the 1 mg/mL HES-CH aqueous solution, 5 mL of a chloroform solution of 1,1′-dioctadecyl-3,3,3′,3′-tetramethylindotricarbocyanine iodide (DIR) was slowly added dropwise into the HES-CH aqueous solution (1 mg/mL); the remaining steps were the same as above.

b. Preparation of radionuclide ¹⁷⁷Lu-labeled antibody: HYNIC and PECAM-1-4G6 were subjected to a reaction for 2 h in an acidic environment, and a resulting reaction product was subjected to filtration and then a reaction with a fresh 177Lu lotion for 30 min in the presence of a reducing agent. A small amount of a resulting reaction product was subjected to radioactive thin-layer chromatography to measure a labeling rate; purification was conducted by a PD-10 column, and a radiochemical purity was measured by the radioactive thin-layer chromatography.

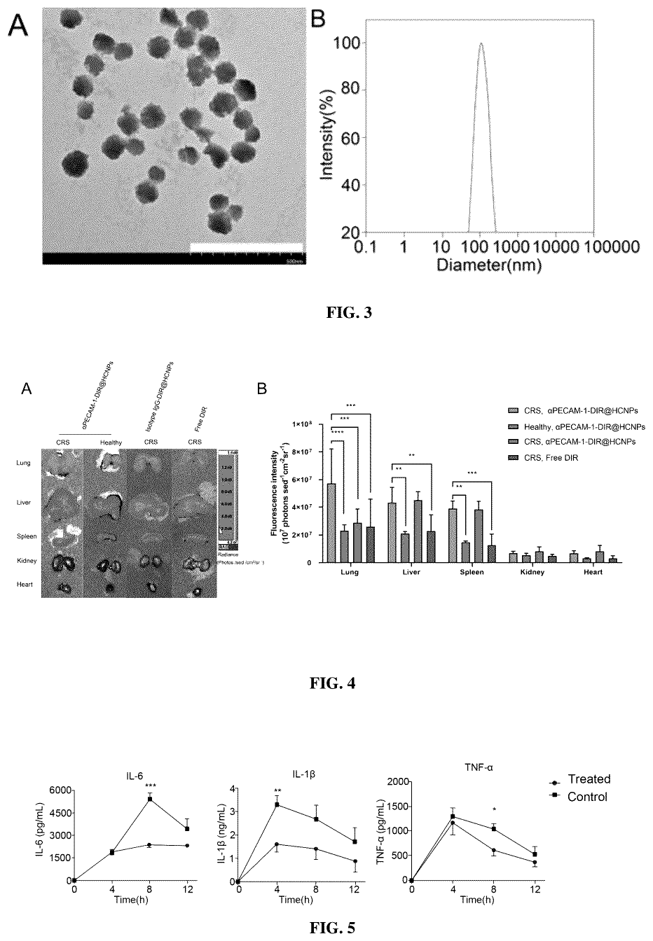

c. Preparation and characterization of ¹⁷⁷Lu-αPECAM-1-AT@HCNPs: DSC was added to the 1 mg/mL HCNPs aqueous solution at room temperature and stirred overnight, dialyzed for 24 h, and then added with 25 μg of the radionuclide-labeled PECAM-1-4G6 mAb to allow reaction by stirring for 3 h at room temperature, to obtain a PECAM-1-HCNPs aqueous solution. Similarly, PECAM-1-DIR@HCNPs were prepared according to the above method (using fluorescein to label the nanoprobe and studying in vivo distribution of the nanoprobe). Antithrombin (AT) and the nanoparticles were dissolved in ultrapure water in an equal proportion and stirred for 2 h, such that the AT was adsorbed and loaded onto the nanoparticles through electrostatic interaction. The αPECAM-1-AT@HCNPs were subjected to ultrafiltration to remove unloaded AT to obtain purified αPECAM-1-AT@HCNPs. The microscopic appearance of the nanoprobe was observed by DLS and TEM (the results were shown in FIG. 3).

### 3. Imaging Specificity of ¹⁷⁷Lu-αPECAM-1-AT@HCNPs

Detection of an inflammatory endothelial imaging function of ¹⁷⁷Lu-αPECAM-1-AT@HCNPs in CRS mice: the ¹⁷⁷Lu-αPECAM-1-AT@HCNPs were injected from the tail vein of model mice, collected images were analyzed by SPECT, highly developed parts were found out by quantitative analysis, and it was evaluated that there was a higher imaging sensitivity of the nanoprobe. After the SPECT was completed, organ tissues such as brain, heart, liver, spleen, lung, and kidney were taken to detect an amount of radionuclide in each organ to understand its biological distribution.

### 4. Evaluation of Endothelial Reconstruction and Anti-Inflammatory Functions of ¹⁷⁷Lu-αPECAM-1-AT@HCNPS

a. After CRS model mice were intervened by ¹⁷⁷Lu-αPECAM-1-AT@HCNPs, a blood cell analyzer was used to detect the haematocrit of the model mice in each group, to measure fluid leakage; the mice were infused with fluorescently labeled low-/high-molecular weight dextran through tail vein, and immunofluorescence was conducted to evaluate macromolecule leakage (the results were shown in FIG. 7).

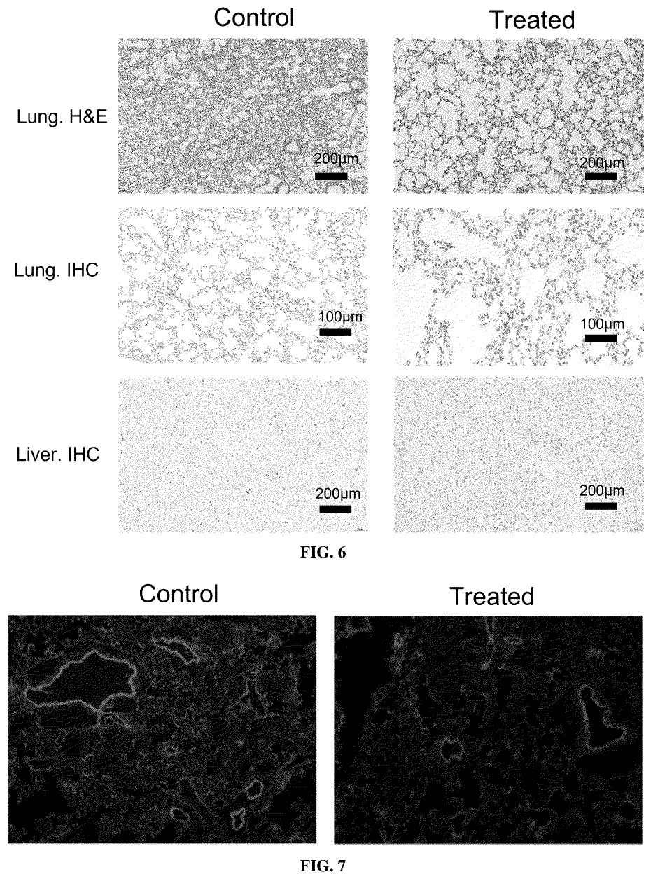

b. After the intervention with ¹⁷⁷Lu-αPECAM-1-AT@HCNPs, the plasma inflammatory factor levels of the model mice in each group were detected (the results were shown in FIG. 5); the leukocyte infiltration in liver, kidney, lung and other organs was compared, and the impact of intervention measures on the inflammatory response in vivo was evaluated. As shown in FIG. 6, the ¹⁷⁷Lu-αPECAM-1-AT@HCNPs could effectively intervene and alleviate the inflammatory response in vivo.

c. Evaluation on the degree of inflammation in tissues and organs: after intervention with ¹⁷⁷Lu-αPECAM-1-AT@HCNPs, the mice were anesthetized and their liver, lungs, and kidneys were collected, the degree of inflammatory reaction in tissues and organs was observed through H&E staining, and immunohistochemistry was conducted to observe granulocyte infiltration in the tissues and organs.

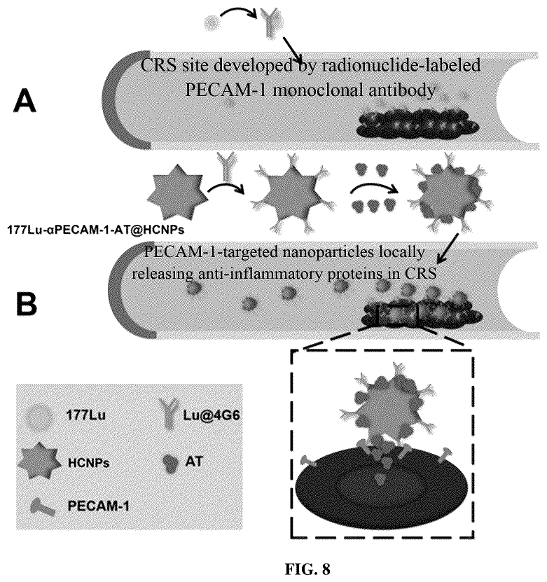

(II) Analysis: CAR-T-related CRS is an acute inflammatory syndrome caused by pro-inflammatory factors acting on the microvascular endothelium of lungs, liver and other organs. Its mechanism is related to damages to the vascular barrier caused by CRS and an increase in soluble platelet endothelial cell adhesion molecule-1 (sPECAM-1) of serum caused by PECAM-1 fragmentation. It is not yet clear whether early monitoring and intervention of CRS can be achieved by using extracellular breakpoints of the PECAM-1 as a target to detect the degree of damage from the CRS to endothelial system. In the present disclosure, a nanoparticle is proposed, which targets PECAM-1, is labeled with a monoclonal antibody (4G6) targeting PECAM-1 IgD6 through radionuclide labeling, and is modified to load AT. This nanoparticle can realize local imaging and inflammation suppression of CRS, thereby providing a novel technology for early monitoring and intervention of CRS. As shown in FIG. 8, the PECAM-1-targeted nanoprobe for integrated diagnosis and treatment was prepared, while a radionuclide 177Lu-labeled antibody 4G6, which had a half-life (6 d to 8 d) in a time window suitable for CAR-T and could target the IgD6 region on PECAM-1, was used to construct ¹⁷⁷Lu-αPECAM-1-ligated HCNPs, which could adsorb a macromolecular drug antithrombin (AT). The ¹⁷⁷Lu-αPECAM-1-AT@HCNPs achieve early capture of target organ imaging with CRS under guidance of the radionuclide ¹⁷⁷Lu-labeled single-chain antibody 4G6. At the same time, the nanoparticles accurately release loaded AT in the localized area of CRS injury, thereby inhibiting excessive coagulation activation and reducing inflammations. These features prove the effectiveness of the nanoparticles involved in the present disclosure for CRS monitoring and treatment.

It will be clear to those skilled in the art that various modifications to the above examples can be made without departing from the general spirit and concept of the present disclosure. These modifications shall all fall within the protection scope of the present disclosure. The claimed protection schemes of the present disclosure shall be determined by the claims.

1. A preparation method of a platelet endothelial cell adhesion molecule-1 (PECAM-1)-targeted nanoprobe for integrated diagnosis and treatment, comprising the following steps:
S1, preparation of radionuclide-labeled PECAM-1_4G6 mAb: subjecting hydrazinonicotinamide (HYNIC) and PECAM-1-4G6 to a reaction in an acidic environment, and filtering a resulting reaction product to allow a reaction with a radionuclide lotion in the presence of a reducing agent to obtain the radionuclide-labeled PECAM-1_4G6 mAb; and
S2, preparation of αPECAM-1-AT@HCNPs: adding N,N′-disuccinimidyl carbonate (DSC) into an aqueous solution of HES-CH nanoparticles (HCNPs), mixing evenly, conducting dialysis, and adding the radionuclide-labeled PECAM-1_4G6 mAb to allow a reaction at a room temperature to obtain an aqueous solution of αPECAM-1-HCNPs; and dissolving the αPECAM-1-HCNPs and antithrombin in an equal proportion, such that the antithrombin is adsorbed and loaded onto the αPECAM-1-HCNPs through an electrostatic interaction, and conducting purification to obtain the αPECAM-1-AT@HCNPs.

2. The preparation method of a PECAM-1-targeted nanoprobe for integrated diagnosis and treatment according to claim 1, wherein the radionuclide is ¹⁷⁷Lu; and the reducing agent comprises 1-ethyl-3-(3-dimethylaminopropyl)carbodiimide hydrochloride (EDC·HCl) and N-hydroxysuccinimide (NHS).

3. The preparation method of a PECAM-1-targeted nanoprobe for integrated diagnosis and treatment according to claim 1, wherein ¹⁷⁷Lu-labeled PECAM-1_4G6 mAb is added and stirred at a room temperature to allow a reaction to obtain an aqueous solution of ¹⁷⁷Lu-αPECAM-1-HCNPs in step S2.

4. The preparation method of a PECAM-1-targeted nanoprobe for integrated diagnosis and treatment according to claim 1, wherein the purification in step S2 specifically comprises: after the antithrombin is adsorbed and loaded onto the αPECAM-1-HCNPs through the electrostatic interaction, ultrafiltration is conducted to remove unloaded antithrombin to obtain purified αPECAM-1-AT@HCNPs.

5. The preparation method of a PECAM-1-targeted nanoprobe for integrated diagnosis and treatment according to claim 1, wherein the αPECAM-1-HCNPs and the antithrombin are dissolved in ultrapure water in the equal proportion, stirred to allow a reaction to electrostatically load the antithrombin in step S2.

6. The preparation method of a PECAM-1-targeted nanoprobe for integrated diagnosis and treatment according to claim 1, wherein a preparation process of the HCNPs comprises: dissolving hydroxyethyl starch-cholesterol conjugated amphiphilic polymer (HES-CH) in water and conducting an ultrasonic treatment to obtain a HES-CH aqueous solution; adding chloroform dropwise into the HES-CH aqueous solution, conducting the ultrasonic treatment while adding the chloroform, and conducting rotary evaporation to remove the chloroform to obtain a HCNPs aqueous solution; and subjecting the HCNPs aqueous solution to centrifugation, discarding a resulting precipitate, and fully freeze-drying to obtain the HCNPs.

7. αPECAM-1-AT@HCNPs prepared by the preparation method according to claim 1.

8-10. (canceled)

11. The αPECAM-1-AT@HCNPs according to claim 7, wherein the radionuclide is 177Lu; and the reducing agent comprises 1-ethyl-3-(3-dimethylaminopropyl)carbodiimide hydrochloride (EDC· HCl) and N-hydroxysuccinimide (NHS).

12. The αPECAM-1-AT@HCNPs according to claim 7, wherein 177Lu-labeled PECAM-1_4G6 mAb is added and stirred at a room temperature to allow a reaction to obtain an aqueous solution of 177Lu-αPECAM-1-HCNPs in step S2.

13. The αPECAM-1-AT@HCNPs according to claim 7, wherein the purification in step S2 specifically comprises: after the antithrombin is adsorbed and loaded onto the αPECAM-1-HCNPs through the electrostatic interaction, ultrafiltration is conducted to remove unloaded antithrombin to obtain purified αPECAM-1-AT@HCNPs.

14. The αPECAM-1-AT@HCNPs according to claim 7, wherein the αPECAM-1-HCNPs and the antithrombin are dissolved in ultrapure water in the equal proportion, stirred to allow a reaction to electrostatically load the antithrombin in step S2.

15. The αPECAM-1-AT@HCNPs according to claim 7, wherein a preparation process of the HCNPs comprises: dissolving hydroxyethyl starch-cholesterol conjugated amphiphilic polymer (HES-CH) in water and conducting an ultrasonic treatment to obtain a HES-CH aqueous solution; adding chloroform dropwise into the HES-CH aqueous solution, conducting the ultrasonic treatment while adding the chloroform, and conducting rotary evaporation to remove the chloroform to obtain a HCNPs aqueous solution; and subjecting the HCNPs aqueous solution to centrifugation, discarding a resulting precipitate, and fully freeze-drying to obtain the HCNPs.
